(19) United States
(12) Patent Application Publication
(10) Pub. No.: US 2026/0158131 A1
(43) Pub. Date: Jun. 11, 2026

(54) ROTAVIRUS B VIRUS-LIKE-PARTICLE COMPOSTIONS AND ANTIBODY ASSAYS

(71) Applicant: University of Kentucky Research Foundation, Lexington, KY (US)

(72) Inventors: Feng Li, Lexington, KY (US); Dan Wang, Lexington, KY (US)

(21) Appl. No.: 19/387,411

(22) Filed: Nov. 12, 2025

Related U.S. Application Data
(60) Provisional application No. 63/719,318, filed on Nov. 12, 2024.

(51) Int. Cl. A61K 39/15; A61K 39/00; A61P 31/14; C07K 14/005; C12N 7/00; G01N 33/68

(52) U.S. Cl. CPC A61K 39/15; A61P 31/14; C07K 14/005; C12N 7/00; G01N 33/6854; A61K 2039/5258; A61K 2039/552; C12N 2720/12322; C12N 2720/12323; C12N 2720/12334; C12N 2720/12351; G01N 2333/14

(57) ABSTRACT

Compositions and methods are provided for immunization against equine rotavirus group B (ERVB). The compositions comprise ERVB structural proteins VP4, VP2, VP6, and VP7, or VP2, VP6, VP7, and a modified VP4, which can be assembled into virus-like particles (VLPs). The invention includes immunogenic compositions and vaccines for administration to non-human animals, including pregnant mares to confer passive immunity to foals. Methods of stimulating immune responses, preventing ERVB spread, conferring passive immunity, evaluating vaccine efficacy, detecting ERVB antibodies, and producing VLPs in baculovirus-infected insect cells are disclosed. The compositions may further include inactivation agents. Nucleotides and vectors encoding the ERVB proteins are also provided.

### RELATED APPLICATIONS

This application claims priority from U.S. Provisional Application Ser. No. 63/719,318 filed Nov. 12, 2025, the entire disclosure of which is incorporated herein by this reference.

### REFERENCE TO AN ELECTRONIC SEQUENCE LISTING

The contents of the electronic sequence listing (Li UK 2858US.xml; Size: 52,232 bytes; and Date of Creation: Nov. 12, 2025) is herein incorporated by reference in its entirety.

### TECHNICAL FIELD

The present disclosure is directed to composition including a virus-like particle for Rotavirus Group B (RVB), including Equine RVB (ERVB). The present disclosure is also directed to and methods for using the composition as a vaccine for RVB, for stimulating an immune response against RVB, for preventing the spread of RVB, and for treating, including prophylactic treatment, RVB. The present disclosure is also directed to antibody assays for RVB.

### INTRODUCTION

Rotaviruses (RVs) belong to the Reoviridae family, which is further divided into two subfamilies: Sedoreovirinae and Spinareovirinae. The Sedoreovirinae subfamily has six genera, with RVs as one of them. Rotavirus was originally identified in mice and vervet monkeys, before its emergence in humans.

RV is a double-stranded RNA virus with a segmented genome under positive polarity. The virus has 11 genome segments, each coding for at least one protein. The RV genome encodes six structural proteins (VP) and six non-structural proteins (NSP). Specifically, segments 1, 2, 3, and 4 code for VP1, VP2, VP3, and VP4, respectively. VP4 is proteolytically cleaved into VP5* and VP8* during viral replication. Segments 6 and 9 express VP6 and VP7. Segment 5 encodes NSP1, while segments 7, 8, and 10 code for NSP3, NSP2, and NSP4. Segment 11 has two open reading frames that express NSP5 and NSP6, respectively. The total genome size is approximately 18.5 kb, with individual genome segment sizes ranging from 667 to 3302 nucleotides in length.

The RV genus has 11 species, also called groups/serogroups, designated as A-D and F-L. These group demarcations are based on the nucleotide sequence of the inner capsid protein VP6. Rotaviruses from groups A, B, and C can infect humans and animals, while D, E, F, and G predominantly infect animals.

Vaccines based on virus-like particles (VLPs) for rotavirus A have been described in the art, including immunogenic compositions comprising rotavirus A VLPs. In contrast, equine rotavirus B is a newly emerging disease that infects foals and causes clinical diarrhea. In addition to horses, other animals can be affected, such as ruminants, including cattle, and pigs. There are currently no vaccines or other compositions available to control or limit the spread of rotavirus B in horses and other agricultural animals and humans. Existing VLP-based vaccines for rotavirus A do not address the unique antigenic and epidemiological features of rotavirus B, including equine rotavirus B.

Accordingly, there remains a need for compositions and methods specifically directed to the prevention and control of rotavirus B infection, including equine rotavirus B infection.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The present invention provides compositions and methods relating to equine rotavirus group B (ERVB) virus-like particles (VLPs) and their use as immunogenic compositions or vaccines for non-human animal subjects.

In certain embodiments, the composition comprises ERVB structural proteins VP4, VP2, VP6, and VP7. In alternative embodiments, the composition comprises ERVB VP2, VP6, and VP7, and a modified ERVB VP4 comprising the sequence of SEQ ID NO: 3.

The composition can be formulated as an immunogenic composition or vaccine for administration to a non-human animal subject. In some embodiments, the non-human animal subject is a mammal, such as a horse, ruminant, swine, or mouse.

In some embodiments, the ERVB VP4 or modified VP4, VP2, VP6, and VP7 are assembled into a virus-like particle (VLP). The VLP may be a triple-layer VLP, as determined by electron microscopy or other biomedical analysis.

The VLP may be antigenically reactive with convalescent serum from an equine subject previously infected with ERVB, indicating that the VLP presents conformational epitopes recognized by antibodies generated during natural infection.

In certain embodiments, the composition further comprises an inactivation agent. In certain embodiments, the composition further comprises an adjuvant.

The ERVB structural proteins may be produced in a baculovirus-infected insect cell expression system.

The invention further provides nucleotides encoding each of ERVB VP4, VP2, VP6, and VP7, or encoding VP2, VP6, VP7, and a modified VP4 comprising SEQ ID NO: 3. Vectors comprising such nucleotides are also provided.

The invention also provides methods of administering the composition to a non-human animal subject in need of protection against ERVB. In some embodiments, the method stimulates an immune response against ERVB in the subject. In other embodiments, the method prevents the spread of ERVB in a non-human animal population by administering the composition to one or more members of the population.

A further aspect of the invention provides a method of conferring passive immunity to a foal, comprising administering the composition to a pregnant mare. In certain embodiments, the composition is administered at least once, twice, or three times during gestation, with each administration separated by about 21 days. In certain embodiments, the composition is administered three times during gestation, with each administration separated by about 21 days.

The invention also provides a method of evaluating vaccine efficacy, comprising administering the composition to a pregnant mare, challenging the resulting foal with ERVB, and measuring viral shedding or infection.

In another aspect, the invention provides a method of detecting antibodies to ERVB in a biological sample, comprising contacting the sample with a VLP as described herein and detecting binding.

The presently-disclosed subject matter also provides a method of producing a VLP, comprising expressing ERVB VP2, VP6, VP7, and VP4 or modified VP4 in a baculovirus-infected insect cell, harvesting, and purifying the VLP.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Viral fecal shedding in foals infected with a low dose equine rotavirus B virus stock with Ct21. The fecal shedding levels were calculated for one MVP foal and one unvaccinated control foal during the first 5 days pos challenge. Two foals were challenged with a low dose equine rotavirus B virus stock with Ct21. Fecal viral shedding levels were analyzed in a RT-qPCR assay with ERVB VP7-specific amplification primers and probe. The Ct. cut-off value for this assay is 40. The viral shedding levels for each foal are summarized. Please note that Ct value—With real-time PCR, the Cycle of Threshold (Ct) is the cycle at which the amplification plot crosses the threshold of detection. Lower Ct numbers indicate more RNA or viruses in a given specimen [i.e., Ct: 20=strong positive; Ct: 35=weak positive]. Ct value 40 will be shown if the specimen does not test positive (no virus shedding).

FIG. 2. Dynamics of viral fecal shedding in foals infected with a high dose equine rotavirus B virus stock with Ct19. The median fecal shedding levels are calculated for each group (n=4 for MVP group and n=6 for control unvaccinated group) during the first 5 days pos challenge. Foals were challenged with a high dose equine rotavirus B virus stock with Ct19. Fecal viral shedding levels were analyzed in a RT-qPCR assay with ERVB VP7-specific amplification primers and probe. The Ct. cut-off value for this assay is 40. The median viral shedding levels for individual group with standard deviation bars are summarized. Please note that Ct value—With real-time PCR, the Cycle of Threshold (Ct) is the cycle at which the amplification plot crosses the threshold of detection. Lower Ct numbers indicate more RNA or viruses in a given specimen [i.e., Ct: 20=strong positive; Ct: 35=weak positive]. Ct value 40 will be shown if the specimen does not test positive (no virus shedding).

FIG. 3A-3D. Baculovirus-expressed MVP triple layer particles (TLPs) consisting of mVP4, VP7, VP6 and VP2. (FIG. 3A) Flag-VP5* and 6×His-VP2 proteins were detected in Western-Blot from Sf9 cells infected with the recombinant baculovirus expressing VP5*(mVP4)/VP7/VP6/VP2 with anti-Flag and anti-His antibodies, respectively. (FIG. 3B) TLPs were detected by Electron Microscope (from Sf9 cells infected with the baculovirus expressing VP5*(mVP4)/VP7/VP6/VP2, 100 nm scale bar). (FIG. 3C) representative image of individual TLPs (50 nm scale bar). (FIG. 3D) Electron microscope detection of equine rotavirus B particles that were purified from feces of equine rotavirus B infected foals (50 nm scale bar).

### BRIEF DESCRIPTION OF THE SEQUENCE LISTING

SEQ ID NO: 1 is the amino acid sequence for VP4 of Equine Rotavirus Group B (ERVB).

SEQ ID NO: 2 is the nucleic acid sequence encoding the amino acid sequence of SEQ ID NO: 1.

SEQ ID NO: 3 is the amino acid sequence for modified VP4 (mVP4) of ERVB.

SEQ ID NO: 4 is the nucleic acid sequence encoding the amino acid sequence of SEQ ID NO: 3.

SEQ ID NO: 5 is the amino acid sequence for VP6 of ERVB.

SEQ ID NO: 6 is the nucleic acid sequence encoding the amino acid sequence of SEQ ID NO: 5.

SEQ ID NO: 7 is the amino acid sequence for VP7 of ERVB.

SEQ ID NO: 8 is the nucleic acid sequence encoding the amino acid sequence of SEQ ID NO: 7.

SEQ ID NO: 9 is the amino acid sequence for VP2 of ERVB.

SEQ ID NO: 10 is the nucleic acid sequence encoding the amino acid sequence of SEQ ID NO: 9.

SEQ ID NO: 11 is the nucleic acid sequence encoding the amino acid sequence of an exemplary ERVB virus-like particle (VLP) provided in accordance with the presently-disclosed subject matter, including VP4, VP6, VP7, and VP2.

SEQ ID NO: 12 is the nucleic acid sequence encoding the amino acid sequence of an exemplary ERVB virus-like particle (VLP) provided in accordance with the presently-disclosed subject matter, including mVP4, VP6, VP7, and VP2.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter includes compositions and methods relating to equine rotavirus group B (ERVB) virus-like particles (VLPs) and their use as immunogenic compositions or vaccines for non-human animal subjects.

In certain embodiments, the composition comprises ERVB structural proteins VP4, VP2, VP6, and VP7. In alternative embodiments, the composition comprises ERVB VP2, VP6, and VP7, and a modified ERVB VP4 comprising the sequence of SEQ ID NO: 3.

As used herein, modified ERVB VP4 refers to a structural protein derived from equine rotavirus group B (ERVB) VP4, wherein the protein comprises a C-terminal fragment corresponding to VP5*(as set forth in SEQ ID NO: 3), rather than the full-length VP4 sequence. Providing a modified VP4 (VP5*) can enhance the immunogenic presentation of key conserved neutralizing epitopes in the VP5* protein among different members of animal rotavirus B species, improve VLP assembly or stability, and/or increase the breadth or potency of the immune response. The VP5* domain is responsible for membrane penetration during viral entry and is a target for broadly neutralizing antibodies. Incorporating VP5* into VLPs can facilitate effective induction of protective immunity.

The composition can be formulated as an immunogenic composition or vaccine for administration to a non-human animal subject. In some embodiments, the non-human animal subject is a mammal, such as a horse, ruminant, swine, or mouse.

As used herein, the term immunogenic composition refers to a composition that comprises one or more antigens and is capable, upon administration to a subject, of eliciting an immune response specific to the antigen(s) present. The immune response may include, but is not limited to, the production of antibodies, activation of T cells, or other cellular or humoral immune mechanisms. The immunogenic composition may further comprise one or more pharmaceutically acceptable carriers, adjuvants, stabilizers, or excipients. As used herein, the term vaccine refers to an immunogenic composition that is administered to a subject with the objective of inducing an immune response that reduces the likelihood, severity, or duration of infection or disease caused by a pathogen, or otherwise provides a beneficial immunological effect. A vaccine may be prophylactic (preventive) or therapeutic, and may be administered to any suitable subject, including non-human animals.

In certain embodiments, the composition is intended for administration to a pregnant mare to confer immunity to a foal. Passive immunity refers to the transfer of protective antibodies from a vaccinated or previously exposed individual to another subject, thereby conferring temporary immune protection without the recipient's own immune system having to mount a primary response. In the context of maternal vaccination, administration of an immunogenic composition or vaccine to a pregnant mare induces the production of specific antibodies against equine rotavirus group B (ERVB). These antibodies are transferred from the mare to the foal, primarily via colostrum and milk, providing the newborn foal with immediate, albeit temporary, protection against ERVB infection during the early period of life when the foal's own immune system is immature. Maternal vaccination is thus an effective strategy for conferring passive immunity to offspring at risk of ERVB exposure.

In some embodiments, the ERVB VP4 or modified VP4, VP2, VP6, and VP7 are assembled into a virus-like particle (VLP). The VLP may be a triple-layer VLP, as determined by electron microscopy or other structural analysis. As used herein, the term triple-layer VLP refers to a virus-like particle (VLP) that comprises three concentric protein layers, structurally analogous to the native rotavirus virion. In the context of ERVB, a triple capsid layer VLP includes an inner capsid layer formed by VP2, a middle capsid layer formed by VP6, and an outer capsid layer formed by VP7 and/or VP4 (or a modified VP4). The triple-layer VLP mimics the morphology and antigenic presentation of native rotavirus particles, as confirmed by electron microscopy and biochemical analysis.

The VLP can be antigenically reactive with convalescent serum from an equine subject previously infected with ERVB, indicating that the VLP presents conformational epitopes recognized by antibodies generated during natural infection.

In certain embodiments, the composition further comprises an inactivation agent. For the inactivation of the baculoviruses expressing VLP, Triton X-100 was added to the baculovirus culture with a final concentration of 1% (v/v) for incubation at 4° C. overnight.

The ERVB structural proteins can be produced in a baculovirus-infected insect cell expression system. The baculovirus vector simultaneously expresses four structural proteins (VP4 or mVP4, VP7, VP6, and VP2) with each under the respective promoter and terminator. In certain embodiments, a nucleotide sequence comprising the sequences of SEQ ID NO: 2, SEQ ID NO: 6, SEQ ID NO: 8, and SEQ ID NO: 10 can be provided in a vector. In certain embodiments, a nucleotide sequence comprising the sequences of SEQ ID NO: 4, SEQ ID NO: 6, SEQ ID NO: 8, and SEQ ID NO: 10 can be provided in a vector. In certain embodiments, the nucleic acid sequence of SEQ ID NO: 11 can be provided in a vector. In certain embodiments, the nucleic acid sequence of SEQ ID NO: 12 can be provided in a vector.

The invention further provides nucleotides encoding each of ERVB VP4, VP2, VP6, and VP7, or encoding VP2, VP6, VP7, and a modified VP4. Vectors comprising such nucleotides are also provided. As used herein the term vector refers to a nucleic acid molecule (such as a plasmid, viral genome, etc.) that is capable of carrying and delivering one or more nucleotide sequences encoding ERVB structural proteins (e.g., VP2, VP4 or mVP4, VP6, VP7) into a host cell, thereby facilitating expression of the encoded proteins. The vector can include regulatory elements such as promoters, enhancers, polyadenylation signals, and selectable markers, and can be suitable for use in a baculovirus-infected insect cell expression system or other recombinant expression systems.

The invention also provides methods of administering the composition to a non-human animal subject in need of protection against ERVB. In some embodiments, the method stimulates an immune response against ERVB in the subject. In other embodiments, the method prevents the spread of ERVB in a non-human animal population by administering the composition to one or more members of the population.

A further aspect of the invention provides a method of conferring passive immunity to a foal, comprising administering the composition to a pregnant mare. In certain embodiments, the composition is administered three times during gestation, with each administration separated by about 21 or 28 days. Each pregnant mare is immunized three times intramuscularly (by the neck) with one injection (˜1 ml each) of the vaccine preparation, respectively, at approximately 8, 9, 10 months of their gestation with a 21-day interval.

The invention also provides a method of evaluating vaccine efficacy, comprising administering the composition to a pregnant mare, challenging the resulting foal with ERVB, and measuring viral shedding or infection. In one embodiment, vaccine efficacy is evaluated by administering the ERVB VLP composition to a pregnant mare according to a defined immunization schedule (e.g., three intramuscular injections at approximately 8, 9, and 10 months of gestation, with 21-day intervals). After parturition, the resulting foal is challenged within 24 hours of birth by oral administration of a defined dose of ERVB viral stock (e.g., low-dose or high-dose, as characterized by RT-qPCR Ct value). Examples of endpoints for efficacy assessment include the following. Viral shedding: a daily collection of fecal samples from the challenged foal, with quantification of ERVB RNA by RT-qPCR. Key metrics include the presence/absence of detectable viral RNA, Ct values over time, duration of shedding, and peak viral load. Clinical signs: Monitoring for clinical diarrhea or other symptoms of ERVB infection. Protection outcomes: Complete protection (no detectable shedding), partial protection (delayed or reduced shedding), or breakthrough infection (shedding comparable to controls). Comparative analysis: Results are compared to foals born to sham-vaccinated mares challenged in parallel.

In another aspect, the present invention provides a method of detecting antibodies to ERVB in a biological sample, comprising contacting the sample with a VLP as described herein and detecting binding. Antibody detection can be performed using methods known in the art, including, for example, ELISA.

The present invention also provides a method of producing a VLP, comprising expressing ERVB VP2, VP6, VP7, and VP4 or modified VP4 in a baculovirus-infected insect cell, harvesting, and purifying the VLP. In brief, Sf9 cells are infected with a recombinant baculovirus expressing mVP4, VP7, VP6, and VP2 proteins. Following 3-day cultivation at 27° C., the culture fluids are inactivated with Triton X-100 at 1% of concentration (v/v) at 4° C. The vaccine is prepared by the addition of adjuvant to the inactivated culture fluids to a final volume of 10%.

As will be appreciated by one of ordinary skill in the art upon study of this document, the presently disclosed compositions comprising equine rotavirus group B (ERVB) structural proteins VP4, VP6, VP7, and VP2, whether individually or assembled into virus-like particles (VLPs), are not found in nature. In particular, the specific combination and recombinant assembly of these proteins, as described herein, do not occur naturally in equine rotavirus B or in any naturally occurring rotavirus particle. Native ERVB virions contain these structural proteins in a specific arrangement and context dictated by viral replication; however, the compositions disclosed herein are produced recombinantly, optionally include modified forms, such as truncated or tagged VP4, and are formulated as immunogenic compositions or vaccines. The resulting VLPs are non-infectious and structurally distinct from wild-type virions, as they are engineered to present selected epitopes and may include modifications to enhance immunogenicity, stability, or safety. Thus, the disclosed compositions represent unique, non-naturally occurring assemblies of ERVB structural proteins, distinguishable from any composition or particle found in nature.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites, and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUBMB Joint Commission on Biochemical Nomenclature (See, iubmb.qmul.ac.uk/).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK® and UNIPROT®. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a,” “an,” and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “non-human animal subject” can be a vertebrate, such as a mammal, a fish, a bird, a reptile, or an amphibian. Thus, the subject of the herein disclosed methods can be a non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Epidemiology, Molecular Biology, and Receptor Characterization of Equine Rotavirus B

The inventors investigated the epidemiology, molecular biology, and receptor biology of Equine Rotavirus B (ERVB) was investigated in equine populations.

Epidemiological Surveillance: Molecular epidemiology studies demonstrated that ERVB was highly prevalent among foals and mares in central Kentucky over multiple foaling seasons. RT-qPCR assays detected ERVB genetic material in mare feces, soil, water, and environmental surfaces at affected premises. Approximately 55% of mare and foal fecal pools tested positive for ERVB, and environmental sampling indicated the virus's stability and persistence in high-traffic areas and on equipment.

Genetic and Phylogenetic Analysis: Sequence analysis revealed that ERVB is closely related to ruminant RVB strains, with >96% protein homology. Evolutionary analysis of the VP7 gene suggested that equine RVB originated from ruminant viruses.

Receptor Biology: ERVB was identified as utilizing sialic acid-containing glycans, specifically polysialic acid (polySia) with α2,8-linkage, as a cellular receptor. Glycan array analysis demonstrated that the ERVB VP8* protein binds polySia with high affinity, in contrast to equine rotavirus A, which utilizes LacDiNAc as a receptor. Binding studies showed that ERVB VP8* binds strongly to polySia-expressing human neuroblastoma cells but not to MA-104 cells, indicating receptor specificity.

### Example 2: Biochemical and Antigenic Characterization of Virus-Like Particle (VLP) Composition Candidates

Virus-like particle (VLP) composition candidates for use as a vaccine for Equine Rotavirus B were prepared and characterized using a baculovirus expression system in insect cells.

Morphological Characterization: Electron microscopy (EM) analysis of wild-type VLPs (WT-VLPs) derived from baculovirus-infected insect cells revealed wheel-like rotavirus particles approximately 100 nm in diameter.

Antigenic Characterization: Western blot analysis using equine convalescent and negative sera identified three unique proteins (VP7, VP6, and VP2) in the VLPs that reacted with positive sera. The data indicated that VP6 and VP7 can be released into the culture medium.

Protein Composition Analysis: Mass spectrometry confirmed the presence of vaccine antigens VP4, VP7, VP6, and VP2 in the WT-VLP vaccine candidate. Quantitative analysis showed that the WT-VLP vaccine was enriched with VP6, followed by VP7, VP4, and VP2.

Modified VLPs: Modified VLPs (mVLPs) were also generated, expressing VP5*(the C-terminal domain of VP4), VP7, VP6, and VP2. EM analysis of mVLPs showed typical triple-layer rotavirus particles. SDS-PAGE analysis confirmed the presence of the expected proteins, with mass spectrometry pending.

Composition/Vaccine Development: Both WT-VLP and mVLP vaccine candidates were advanced for safety and efficacy testing in mouse and horse trials, to identify top candidates for evaluation in pregnant mares and foals.

### Example 3: Sequences

RV is a double-stranded RNA virus with segmented genomes under positive polarity. The virus has 11 genome segments each coding for at least one protein. RV genome codes for 6 structural proteins (VP) and 6 non-structural proteins (NSP). Specifically, segments 1, 2, 3, and 4 code for VP1, VP2, VP3, and VP4, respectively. VP4 is proteolytically cleaved into VP5* and VP8* during viral replication. Segments 6 and 9 express VP6 and VP7. Segment 5 encodes NSP1, while segments 7, 8, and 10 code for NSP3, NSP2, and NSP4. Segment 11 has two open reading frames that express NSP5 and NSP6, respectively. The total genome size is approximately 18.5 kb with individual genome segment sizes ranging from 667 to 3302 nucleotides in length.

Examples of sequences that can be used in accordance with the present invention are as follows. An exemplary equine rotavirus group B (ERVB) virus like particle (VLP) can include: VP4 (SEQ ID NO: 1), VP6 (SEQ ID NO: 5), VP7 (SEQ ID NO: 7), and VP2 (SEQ ID NO: 9). An exemplary ERVB modified VLP (mVLP or MVP) can include: mVP4 (SEQ ID NO: 3), VP6 (SEQ ID NO: 5), VP7 (SEQ ID NO: 7), and VP2 (SEQ ID NO: 9).

### Example 4: Production and Visualization and Analysis of Triple-Layer VLPs and mVLPs

Supernatants from Sf9 cells infected with a baculovirus expressing mVP4 (VP5*)/VP7/VP6/VP2 are clarified by a brief centrifugation at 4° C. at 2,000 rpm for 10 minutes. The clarified supernatants are loaded into 20% sucrose cushion for an ultracentrifugation at 28,000 rpm for 2 hours. The pellets are resuspended with PBS buffer followed by Western-blot analysis of mVP4 (VP5*), containing a Flag epitope tag, and VP2 with a Histidine tag. The pellets are also analyzed by Electron Microscope. The data show that m VLPs resemble wild-type triple layer particles in morphology and their proteins are expressed.

A baculovirus vector expression system (BYES) was used to express virus-like-particles and modified virus-like-particles. These virus-like-particles resemble native equine rotavirus B's particles but are not infectious.

Several lines of evidence were collected in support of the utility of these virus-like-particles (VLP) for inducing an immune response and/or as vaccines for equine rotavirus B. First, the compositions were observed to possess rotavirus-like particles by Electron Microscope (EM). Second, the compositions were found to be reactive to convalescent serum samples from horses that were previously infected with equine rotavirus B. Third, evidence obtained from mass spectrometry analysis indicated the compositions contain viral structural proteins (VP2, VP6, VP7, and VP4 or VP5*) needed for virus-like-particle formation.

Mice and horses have been immunized with two virus-like-particle vaccine candidates for determining their ability in inducing equine rotavirus B specific antibody responses. It is contemplated that these experiments will show induction of protective antibody responses. The compositions are also contemplated for use in ruminants, such as cattle and other small ruminants.

### Example 5: Composition/Vaccine Formulation and Immunization of Pregnant Mares

A modified virus-like-particle (mVLP) vaccine was prepared by infecting insect Sf9 cells with a recombinant baculovirus expressing four structural proteins of equine rotavirus B: VP5*, VP7, VP6, and VP2. At 72-96 h post-infection, infected cells together with culture media were harvested. After 3 cycles of freezing and thawing, culture media were centrifuged at 5,000 for 20 min at 4° C. and the supernatants containing immunogens were collected for vaccine formulation. Supernatants were inactivated with 1% Triton X-100 (v/v) at 4° C. (refrigerator) overnight. Upon the validation of no detectable replicating baculoviruses in Sf9 cells, the inactivated supernatants were mixed with adjuvant to formulate mVLP vaccine that was tested for efficacy in this study. The sham (mock) vaccine was prepared in the same manner except PBS replacing m VLP immunogen.

Among 12 pregnant mares, 7 mares were assigned into sham-vaccinated control group, while the other 5 mares were placed in m VLP vaccination group. Pregnant mares in each vaccination group were immunized three times intramuscularly (by the neck) with one injection (˜1 ml each) of the vaccine preparation, respectively, at approximately 8, 9, 10 months of their gestation with a 21-day interval. The sham (mock) vaccination group was immunized with the same regime using the PBS-adjuvant formulation. Pre-vaccination and post-vaccination sera from all the mares were collected and used for analyzing antibody responses induced by m VLP vaccine. There were no adverse reactions observed in mares receiving vaccinations. Vaccinated mares behaved normally in temperatures and other manners.

### Example 6: Foal Infection and Vaccine Efficacy

A vaccine efficacy study was conducted on foals born to vaccinated mares, which included two phases. Phase I involved a foal challenge experiment with a low-dose equine rotavirus B stock with a Ct value of 21. The Ct (cycle threshold) of PCR reaction refers to the number of cycles needed to replicate enough viral genome RNA to be detected (crosses a threshold line). Higher Ct corresponds to lower virus load and vice versa. Equine rotavirus B is completely refractory to cell culture and there is no cell culture method that can replicate equine rotavirus B so the number of infectious particles could not be quantitated. However, it was estimated, based on the conversion from RT-qPCR derived Ct values, which can be extrapolated from the formula relating Ct value to the amount of infectious virus particles for equine rotavirus A in a culturable system, that the low-dose equine rotavirus stock with Ct21 contained approximately 3×10⁶ infectious particles per ml. Phase II involved a foal challenge study with high-dose equine rotavirus B stock with Ct19, which contained approximately 8×10⁶ infectious particles per ml.

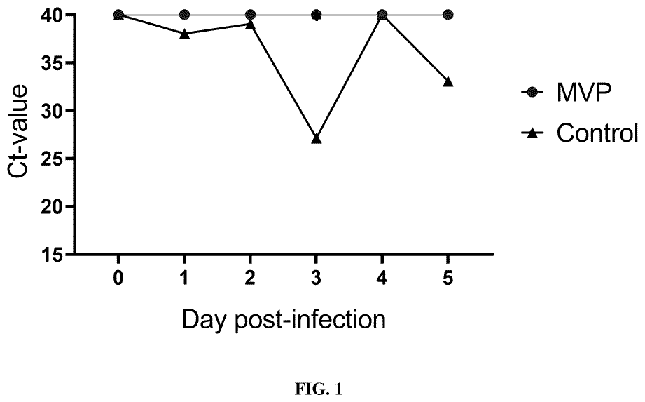

Phase I only had one foal born to an m VLP vaccinated mare and one foal from a sham-vaccinated mare. In this study, two foals were experimentally challenged within the 24 hours of age. Each foal was infected orally with 1 ml of low-dose virus stock with Ct21 through a sterile nasal gastric tube followed by 20 mL of flush solution. As summarized in Table 1 and FIG. 1, the control foal (Y4) born to the sham-vaccinated mare started to shed at 1 day-post-infection (DPI) with most shedding detected at 3 DPI (Ct27). In contrast, Y5 foal born to the m VLP-vaccinated mare was completely protected as no viral shedding was detected in this foal during this study.

TABLE 1
Summary of viral shedding from foals born to MVP-vaccinated and sham-vaccinated
control mares following challenge at 1 day of life with low-dose equine rotavirus
B stock with Ct value 21 (Ct, cycle threshold in PCR reaction)

| Foal ID | 0 DPI | 1 DPI | 2 DPI | 3 DPI | 4 DPI | 5 DPI | 6 DPI | 7 DPI | 8 DPI | 9 DPI | 10 DPI |
|---|---|---|---|---|---|---|---|---|---|---|---|
| Control/Y4 | Neg | 38.3 | 39.09 | 27.13 | Neg | 33.06 | 32.82 | Neg | 39.06 | Neg | Neg |
| MVP/Y5 | Neg | Neg | Neg | Neg | Neg | Neg | Neg | | | | |

Note:
neg (negative) means not detected by RT-qPCR assay.
Ct value: With real-time PCR, the Cycle of Threshold (Ct) is the cycle at which the amplification plot crosses the threshold of detection. Lower Ct numbers indicate more RNA or viruses in a given specimen, e.g., Ct: ~20 = strong positive; Ct: ~35 = weak positive. No Ct value will be shown if the specimen does not test positive.

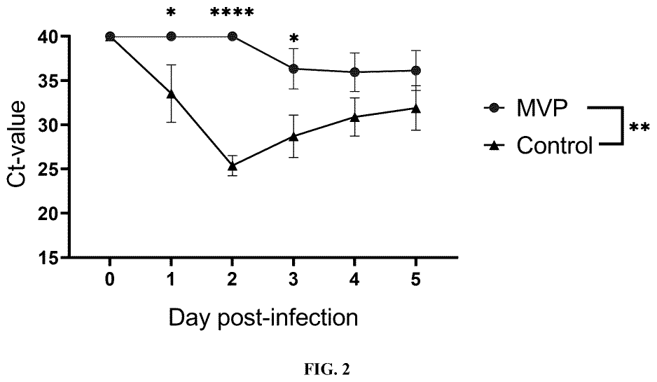

Phase II had six foals born to the sham-vaccinated mare group and 4 foals born to the m VLP-vaccinate mares. Phase II study used a high-dose equine rotavirus B stock with Ct19. The same oral challenge route was used for infecting foals in both groups. As summarized in Table 2 and FIG. 2, all control foals shed at 1 or 2 DPI with starting Ct 22-24 and such low Cts lasting for 4-5 days in most control foals. One outlier control foal Y32 only showing low shedding at 1 and 2 DPI with Cts 30-33.

Four m VLP foals were protected from high-dose (Ct19) equine rotavirus B infection in Phase II study (Table 2 and FIG. 2), which is like the phase I study using low-dose (Ct21) equine rotavirus B. Among five foals, one mVLP foal, Y6, was completely protected with no viral shedding detected during this experiment. Three mVLP foals, Y22, Y25, and Y30, shed lower level of viruses exhibiting Ct values around 33. Foal Y22 only shed lower levels of the virus for 2 days. Overall, viral shedding in m VLP foals was delayed for at least 3 or 4 days when compared to control foals.

TABLE 2
Summary of viral shedding from foals born to MVP-vaccinated and sham-vaccinated
control mares following challenge at 1 day of life with high-dose equine
rotavirus B stock with Ct value 19 (Ct, cycle threshold in PCR reaction)

| Foal ID | 0 DPI | 1 DPI | 2 DPI | 3 DPI | 4 DPI | 5 DPI | 6 DPI | 7 DPI | 8 DPI |
|---|---|---|---|---|---|---|---|---|---|
| Control/Y9 | Neg | 23.49 | 25.2 | 27.63 | 29.9 | 32.18 | 33.01 | 35.7 | 37.64 |
| Control/Y12 | Neg | 23.99 | 25.7 | 28.38 | 25.67 | 25.87 | 27.29 | 34.87 | 34.79 |
| Control/Y13 | Neg | Neg | 23.68 | 22.67 | 28.92 | 25.89 | 32.48 | 32.24 | 31.92 |
| Control/Y21 | Neg | Neg | 24.03 | 27.35 | 33.9 | 38.46 | 39.22 | 36.17 | 34.8 |
| Control/Y23 | Neg | Neg | 22.99 | 26.52 | 26.94 | 29 | 31.48 | 31.18 | 32.53 |
| Control/Y32 | Neg | 33.74 | 30.72 | Neg | Neg | Neg | | | |
| MVP/Y6 | Neg | Neg | Neg | Neg | 39.45 | Neg | Neg | | |
| MVP/Y22 | Neg | Neg | Neg | Neg | Neg | 33.14 | 36.61 | Neg | Neg |
| MVP/Y25 | Neg | Neg | Neg | 34.79 | 32.47 | 31.42 | 34.67 | 35.94 | 36.1 |
| MVP/Y30 | Neg | Neg | Neg | 30.54 | 31.88 | Neg | 34.35 | 34.75 | 33.4 |

| Foal ID | 9 DPI | 10 DPI | 11 DPI | 12 DPI | 13 DPI | 14 DPI | 15 DPI | 16 DPI |
|---|---|---|---|---|---|---|---|---|
| Control/Y9 | Neg | Neg | | | | | | |
| Control/Y12 | Neg | Neg | Neg | | | | | |
| Control/Y13 | 33 | 34.85 | 36.41 | 36.63 | Neg | Neg | | |
| Control/Y21 | Neg | 38.11 | Neg | Neg | Neg | | | |
| Control/Y23 | 37.14 | Neg | Neg | | | | | |
| Control/Y32 | | | | | | | | |
| MVP/Y6 | | | | | | | | |
| MVP/Y22 | Neg | Neg | 33.14 | 36.61 | Neg | Neg | | |
| MVP/Y25 | 35.7 | 35.94 | 35.99 | 33.92 | 36.27 | 38.09 | Neg | Neg |
| MVP/Y30 | 38.29 | Neg | 35 | Neg | 36.78 | Neg | Neg | Neg |

Note:
neg (negative) means not detected by Rt-qPCR assay.
Ct value—With real-time PCR, the Cycle of Threshold (Ct) is the cycle at which the amplification plot crosses the threshold of detection. Lower Ct numbers indicate more RNA or viruses in a given specimen [i.e., Ct: 20 = strong positive; Ct: 35 = weak positive]. No Ct value will be shown if the specimen does not test positive.

The results of this vaccine efficacy study demonstrated that m VLP vaccine can provide protection to foals born to vaccinated mares against equine rotavirus B challenge. Two foals were completely protected against equine rotavirus B infection (Tables 1 and 2, and FIGS. 1 and 2). One foal only showed transient viral shedding for two days post infection. For two foals showing the viral breakthrough infection, viral shedding was delayed for at least 3 days. Viral shedding in vaccinated dams-derived foals was reduced significantly by more than 3-4 logs when compared to unvaccinated and challenged foals, based on the conversion of Ct values to the numbers of infectious particles.

### Example 7: Baculovirus-Expressed mVLP Triple Layer Particles (TLPs) Consisting of mVP4, VP7, VP6 and VP2

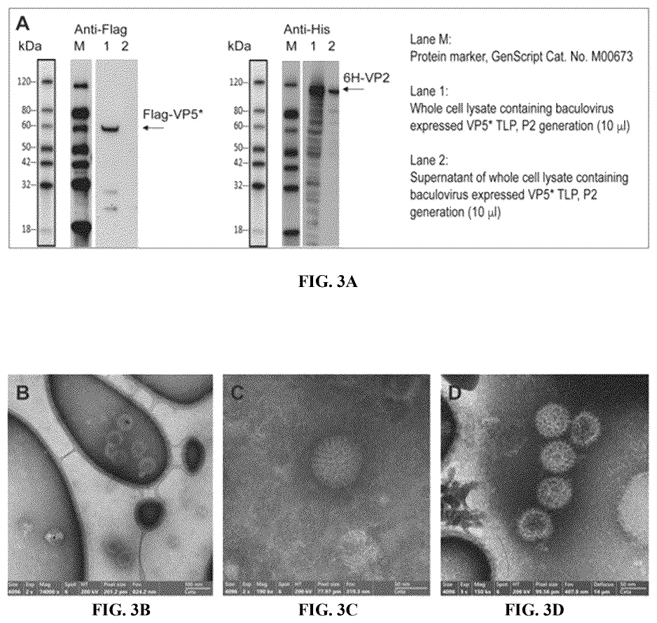

Triple-layered particles (TLPs) comprising mVP4, VP7, VP6, and VP2 were produced using a baculovirus expression system. Sf9 insect cells were infected with a recombinant baculovirus encoding VP5*(mVP4), VP7, VP6, and VP2. Expression of Flag-tagged VP5* and 6×His-tagged VP2 was confirmed by Western blot analysis using anti-Flag and anti-His antibodies, respectively (FIG. 3A). Following infection, TLPs were visualized by transmission electron microscopy, revealing the presence of virus-like particles with a diameter of approximately 100 nm (FIG. 3B). A representative image of individual TLPs is shown in FIG. 3C, with a scale bar of 50 nm. For comparison, equine rotavirus B particles purified from fecal samples of infected foals were similarly visualized by electron microscopy, as shown in FIG. 3D (50 nm scale bar).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A composition, comprising: equine rotavirus group B (ERVB) structural protein (VP) 4, ERVB VP2, ERVB VP6, and ERVB VP7; or a composition comprising ERVB VP2, ERVB VP6, and ERVB VP7, and a modified ERVB VP4 comprising the sequence of SEQ ID NO: 3.

2. The composition of claim 1, provided as an immunogenic composition or vaccine for administration to a non-human animal subject.

3. The composition of claim 2, wherein the non-human animal subject is a mammal.

4. The composition of claim 3, wherein the non-human animal subject is a horse, a ruminant, or a swine.

5. The composition of claim 4, for administration to a pregnant mare to confer immunity to a foal.

6. The composition of claim 1, wherein the ERVB VP4 or ERVB mVP4, ERVB VP2, ERVB VP6, and ERVB VP7 are assembled into a virus-like particle (VLP).

7. The composition of claim 6, wherein the VLP is a triple-layer VLP.

8. The composition of claim 6, wherein the VLP is antigenically reactive with convalescent serum from an equine subject previously infected with ERVB.

9. The composition of claim 1, and further comprising an inactivation agent.

10. The composition of claim 1, wherein the ERVB structural proteins are produced in a baculovirus-infected inset cell expression system.

11. A nucleotide, comprising: a nucleotide encoding each of ERVB VP4, ERVB VP2, ERVB VP6, and ERVB VP7; or a nucleotide encoding each of ERVB VP2, ERVB VP6, and ERVB VP7, and a modified ERVB VP4 comprising the sequence of SEQ ID NO: 3.

12. A vector comprising the nucleotide of claim 11.

13. A method, comprising administering the composition of claim 1 to a non-human animal subject in need of protection against ERVB.

14. A method of stimulating an immune response against ERVB in a non-human animal subject, comprising administering the composition of claim 1 to a non-human animal subject in need of protection against ERVB.

15. A method of preventing the spread of Equine Rotavirus Group B in a non-human animal population, comprising administering to a subject that is a member of the non-human animal population the composition of claim 1.

16. A method of conferring passive immunity to a foal, comprising administering the composition of claim 1 to a pregnant mare.

17. The method of claim 16, and further comprising administering the composition at least three times during gestation, with each administration separated by about 21 days.

18. A method of evaluating vaccine efficacy, comprising administering the composition of claim 1 to a pregnant mare, challenging the resulting foal with ERVB, and measuring viral shedding or infection.

19. A method of detecting antibodies to ERVB in a biological sample, comprising contacting the sample with a VLP of claim 6 and detecting binding.

20. A method of producing a VLP comprising expressing ERVB VP2, VP6, VP7, and VP4 or mVP4 in a baculovirus-infected insect cell, harvesting, and purifying the VLP.


SEQUENCE LISTING (12 sequences)

SEQ ID NO: 1 - protein, 769 aa, Rotavirus B (equine)
MDYKDDDDKGSGISYLRREWQSYGETVRETFEDEDAVAARNGSDTTSERDVPTKADGRYCYKAEVNKSNYTTEVRGFALGEADERVSAEQFQVYSGEVTNGYTFINGDPPCQTVIRIYLNVTGEITIDGQSVQGIYFEATSCIRTDNGYNLHAYRDRSVVPTEAQWKLITRAFSKNGCTGLDGNGQLSASVSIKNEKEWSYDISGETELNMYSWSDLCQQSRTTMSNAEQSSRIIIYEQEDGFWKILTETLWIKLKPYFKPYGTMGGAFKNWLVDSGFEKYEYSYTYTRDGKVVNATTVTYPKPTGKAGINQSWRPATDYNGQYVCMQPGDIFSVWYFEDRWQINQAIYAKNFQSDSRAEGELENTGGLIFRMNYIPSLAGIRNKAGKVKYRYINGGFAQVDAGSYTGMAIILNFECYGRKFYADSNNYPVDNTLNPYICYIGDDYTVGGTHYRNGACSGYAAGYDDTILEHDMTISYTVMKPSDPDFVTGGESYGQSITSGLEVSIRNLQDQINSILAELNIQQVTSAVFSAVTSVGDLPNLFSNVTKIFSKTKDALAKLKGRKVAKTEPVKATMIIDKSNIDVPNVSIVNKMPEEYELGVIYNSMRQARLQREGKHDFPTFALATEMKLPYIQNTNTLTPKFKKYLSDRGLLCDDTAAIQFDPMDLTFSTLRKRNADVLKYKIDPEIAHEVLSEMSTTATRSLFSLNVRKQISTNNEFGSPTYEQIINRILDDREILDIMGKLNRQTVGNLFQEFLDRTKDMLSNYV

SEQ ID NO: 2 - DNA, 2309 bp, Rotavirus B (equine)
  source 1..2309: mol_type = other DNA
atggattacaaggatgacgatgacaagggttctggaatcagttacttgcgtagggagtggcaaagttacggtgaaacggtgcgcgaaaccttcgaagacgaagatgccgtagcggcaagaaacggttccgataccacgtctgagagagatgtacctaccaaggccgatggccgctactgttataaggcagaagtgaacaagtccaattatacaactgaggtaagaggttttgcacttggagaggcggatgaacgtgtatcagctgaacagtttcaggtctattccggagaagtgactaatggatacacctttatcaacggagacccaccttgtcaaacagtaattagaatatatttgaacgttaccggcgaaataactatcgatggacaatcagttcagggtatatactttgaagcgacgtcctgcattagaactgataatggctacaacctgcacgcctaccgtgacaggtctgtggttcccactgaagcgcagtggaaactcataactagggctttttcgaagaatggatgcactggtttggatggcaacggccaactgtcggcgtcagtcagcattaagaacgaaaaggaatggagctacgacataagtggagagactgaactgaatatgtattcctggagcgatctttgccagcaatcgaggacaactatgtctaatgcagagcagtccagcaggatcataatctatgaacaagaagacggattttggaaaattctcactgaaacactttggattaagctcaagccatactttaagccatatggtacaatgggcggagcgttcaagaactggttggttgatagcggatttgagaaatacgagtattcgtatacctatacaagggatggcaaggtagtcaacgcaacaaccgttacttatcccaagccgacaggcaaggcgggaatcaaccagtcctggcgtcccgctaccgactacaatggtcagtacgtctgtatgcagccaggagatatattttcggtatggtattttgaggacagatggcaaattaaccaggctatatacgcaaaaaatttccagtcggactctagggccgaaggtgagctcgaaaatactggtggtctcatttttcgcatgaattatataccctctctggctggcataaggaataaggcgggaaaagtgaagtacagatacattaatggcggcttcgctcaagtggacgccggtagttacacgggtatggctatcatccttaacttcgagtgctacggtagaaaattttacgcggacagcaacaactatcctgtggataacacccttaacccttatatctgctacataggtgatgactacacagttggtggcactcactaccgcaacggagcgtgttcgggctacgccgctggttacgacgatacgatactggagcacgacatgactataagctacactgtaatgaagccttctgatcctgatttcgtaaccggcggcgaatcatacggacaaagcataacctcaggactggaggtcagcataaggaatctccaggatcagatcaactcaatcttggccgagctgaatatccaacaagttacctcagcggttttctccgccgtgacctcagtcggtgatctccctaatctgttttcaaatgtgacgaaaattttttcgaaaacgaaggacgcgctcgccaaactgaagggtcgcaaagtcgccaagaccgaacctgtgaaagcgacgatgattattgacaagtctaacatagacgtgcccaacgtcagcatcgttaacaaaatgcccgaagaatatgaactgggtgtgatatataactcaatgaggcaagcgcgcctccagcgcgagggaaagcatgacttccccacgtttgcgcttgctaccgaaatgaagctcccatatatacaaaacacaaacacgttgactcctaaattcaaaaagtatctcagtgaccgtggtttgctgtgtgacgacacggctgctattcaattcgatccaatggatttgacattcagcactcttagaaagcgtaacgcagacgttttgaaatataagatcgaccctgaaatagcacacgaagtacttagcgaaatgtcgacgacggcaaccaggtcactcttttcccttaacgtaaggaaacagatttcaactaataacgaattcggaagtccaacgtatgagcagatcattaaccgtattcttgatgatcgcgagatccttgacatcatgggaaagcttaatcgccaaaccgttggtaatctgtttcaggaattccttgataggactaaagatatgttgagcaattatgtcta

SEQ ID NO: 3 - protein, 560 aa, synthetic construct
MDYKDDDDKGSGRTTMSNAEQSSRIIIYEQEDGFWKILTETLWIKLKPYFKPYGTMGGAFKNWLVDSGFEKYEYSYTYTRDGKVVNATTVTYPKPTGKAGINQSWRPATDYNGQYVCMQPGDIFSVWYFEDRWQINQAIYAKNFQSDSRAEGELENTGGLIFRMNYIPSLAGIRNKAGKVKYRYINGGFAQVDAGSYTGMAIILNFECYGRKFYADSNNYPVDNTLNPYICYIGDDYTVGGTHYRNGACSGYAAGYDDTILEHDMTISYTVMKPSDPDFVTGGESYGQSITSGLEVSIRNLQDQINSILAELNIQQVTSAVFSAVTSVGDLPNLFSNVTKIFSKTKDALAKLKGRKVAKTEPVKATMIIDKSNIDVPNVSIVNKMPEEYELGVIYNSMRQARLQREGKHDFPTFALATEMKLPYIQNTNTLTPKFKKYLSDRGLLCDDTAAIQFDPMDLTFSTLRKRNADVLKYKIDPEIAHEVLSEMSTTATRSLFSLNVRKQISTNNEFGSPTYEQIINRILDDREILDIMGKLNRQTVGNLFQEFLDRTKDMLSNYV

SEQ ID NO: 4 - DNA, 1683 bp, synthetic construct
  source 1..1683: mol_type = other DNA
atggattacaaggatgacgatgacaagggttctggaaggacaactatgtctaatgcagagcagtccagcaggatcataatctatgaacaagaagacggattttggaaaattctcactgaaacactttggattaagctcaagccatactttaagccatatggtacaatgggcggagcgttcaagaactggttggttgatagcggatttgagaaatacgagtattcgtatacctatacaagggatggcaaggtagtcaacgcaacaaccgttacttatcccaagccgacaggcaaggcgggaatcaaccagtcctggcgtcccgctaccgactacaatggtcagtacgtctgtatgcagccaggagatatattttcggtatggtattttgaggacagatggcaaattaaccaggctatatacgcaaaaaatttccagtcggactctagggccgaaggtgagctcgaaaatactggtggtctcatttttcgcatgaattatataccctctctggctggcataaggaataaggcgggaaaagtgaagtacagatacattaatggcggcttcgctcaagtggacgccggtagttacacgggtatggctatcatccttaacttcgagtgctacggtagaaaattttacgcggacagcaacaactatcctgtggataacacccttaacccttatatctgctacataggtgatgactacacagttggtggcactcactaccgcaacggagcgtgttcgggctacgccgctggttacgacgatacgatactggagcacgacatgactataagctacactgtaatgaagccttctgatcctgatttcgtaaccggcggcgaatcatacggacaaagcataacctcaggactggaggtcagcataaggaatctccaggatcagatcaactcaatcttggccgagctgaatatccaacaagttacctcagcggttttctccgccgtgacctcagtcggtgatctccctaatctgttttcaaatgtgacgaaaattttttcgaaaacgaaggacgcgctcgccaaactgaagggtcgcaaagtcgccaagaccgaacctgtgaaagcgacgatgattattgacaagtctaacatagacgtgcccaacgtcagcatcgttaacaaaatgcccgaagaatatgaactgggtgtgatatataactcaatgaggcaagcgcgcctccagcgcgagggaaagcatgacttccccacgtttgcgcttgctaccgaaatgaagctcccatatatacaaaacacaaacacgttgactcctaaattcaaaaagtatctcagtgaccgtggtttgctgtgtgacgacacggctgctattcaattcgatccaatggatttgacattcagcactcttagaaagcgtaacgcagacgttttgaaatataagatcgaccctgaaatagcacacgaagtacttagcgaaatgtcgacgacggcaaccaggtcactcttttcccttaacgtaaggaaacagatttcaactaataacgaattcggaagtccaacgtatgagcagatcattaaccgtattcttgatgatcgcgagatccttgacatcatgggaaagcttaatcgccaaaccgttggtaatctgtttcaggaattccttgataggactaaagatatgttgagcaattatgtctaa

SEQ ID NO: 5 - protein, 391 aa, Rotavirus B (equine)
MDLIETVNACVRLQKRVLSLAPNTNLNTAGQSILNDYNAIASRVNGKTYALLDQTAILSPYTIHAPIISLAVRISTDDYDDMRNGVESILDCLAAAIRTEGSRPVRVIERRVIEPVVKQLVEDLKLKSLISEISIANFAATDTALIQPEVVETENPLIVGIIEQVVVRQPASLNGGNIRAAIGRWSGNKGSVTCVSGMEAEHMFFVELKARTCGVLNVVYLPAPGVIMVPMPQGRNRESVILDVSAEMTADDFIIDFFDDNNIVHTERGVGLFSFPMCTRIRFRVTPWTQQKSQNGLDTPSLATWANGTSPRQPAVSFMFELRRTFTENDYKFVSRCTSKVQYILETNFPETSFINRPQIEWNVQEMITSDTDTVWSRKIAMLVAAFAAKI

SEQ ID NO: 6 - DNA, 1176 bp, synthetic construct
  source 1..1176: mol_type = other DNA
atggatttgatagaaacggtgaatgcttgtgtgagattgcagaaaagggtacttagtcttgctcccaacacaaacctgaatacggcgggtcagagtatcctcaacgattataatgcgatagcttcgcgcgtcaatggcaaaacgtatgcactgctcgatcaaacagccatactgtcaccatacaccatacatgcacccattatcagtttggctgtccgtatatcaaccgatgattacgatgatatgcgcaacggtgtagagagcatactggactgtctggccgccgcgatcagaacggagggatcacgtccggttagggtgattgagagacgcgtgatcgaacctgttgtaaaacaattggtggaggacttgaagctgaagtcgttgataagcgagataagtatagcgaactttgccgccacggacaccgctttgatccaacctgaggttgtggagacggagaacccactgattgtcggcattattgagcaagtcgtagtccgccagcctgccagcttgaacggaggaaacatcagggccgccatcggcaggtggtccggcaacaaaggtagtgtaacctgtgtatctggcatggaagcggaacacatgttcttcgtagaattgaaagctcgcacgtgcggagtgttgaatgtggtatacctcccggcacctggcgtcatcatggtccccatgccgcagggtagaaatagggagtcagttattcttgatgttagtgcagaaatgacggctgacgactttattattgattttttcgatgacaataatatcgtgcatacagaacgcggcgtgggcttgtttagctttccaatgtgcaccagaataaggtttcgtgtaaccccctggacgcaacagaaatctcagaatggcttggatacgccttccctggcgacatgggcgaatggtacgtcgccgaggcaacctgccgtatccttcatgtttgagcttagaagaacgtttaccgagaacgactataagttcgtgtcaagatgcacttcaaaggttcagtatatacttgagaccaacttccctgagacatcattcataaacagacctcaaatagaatggaatgttcaagaaatgataacaagtgatacggacaccgtgtggtcgagaaaaatagcaatgctggtggcagcctttgctgctaagatatga

SEQ ID NO: 7 - protein, 247 aa, Rotavirus B (equine)
MALPLLLVFAACAKAQLVITPISNPEICVLHASDWNVNSFGDNFTNIFETYNSVTLSFYQYDSTNYDVIDIISKRDYSLCHILAIDVIRPEMDFITFLQSNNECSKYAGQKIHYQKLSTNEEWFVYSKNLKFCPLSDSLIGLYCDTQINGTYFPLSENEKYDVTDLPEFTEMGYVFYSNDDFYICKRINEDKWVNYHLFYREYSASGTVSRAISWDNVWTGFKTFAQVVYKILDIFFNNRRNFEPRA

SEQ ID NO: 8 - DNA, 744 bp, Rotavirus B (equine)
  source 1..744: mol_type = other DNA
atggcactcccattgcttctggtttttgctgcttgtgcgaaggcccaactggttataacgcccatatcaaatccagaaatatgcgtcctgcacgcctctgattggaatgtaaattccttcggcgacaattttacgaatattttcgaaacctataacagtgtaacgctctccttctatcagtacgatagcacaaactatgacgtgattgatataatctctaagagagactactcgctgtgtcacatcttggctatcgatgtaattcgtcccgaaatggatttcatcacttttctccaatccaacaacgagtgtagcaaatatgccggtcaaaagatccactaccaaaaactgtccaccaacgaagaatggttcgtatattccaagaacctgaagttttgccctctcagtgacagtctgattggtctctactgtgacacacagataaacggcacctatttccctctgtcagaaaatgaaaaatatgacgtcaccgacctcccggagtttaccgaaatgggttatgtcttttattctaacgacgatttctacatttgtaagaggatcaatgaagacaagtgggtcaattatcacctgttctatagagaatactccgcgagtggaactgtatcaagggctatatcatgggacaacgtatggacgggctttaagacatttgctcaagtagtttataagatactggacatattttttaacaatcgtagaaacttcgagcctcgtgcctaa

SEQ ID NO: 9 - protein, 954 aa, Rotavirus B (equine)
MMHHHHHHGSGLVPRGSRPGDLDLITSASQAIQATNDKNDKQKIFDQLVNDLKRLSKGVLQPDVQSKLLELSNINGLVFNVEVQEDVVNTINDQPDPTSIFTQNVFQLRTTLSKTLFIDVDAEEYSVYVPLEVQNISPVVIDIRPIQTYKPKTLMYKDTAIIPSNNDLVSDQWGADEILYDSHMFNDINIGQIEDFETYLLEKAVEIKDSLPNINHISQLSKDTNPFNVHNTLCLDFGQKEYYNLISDRTNQSFVGRRQAVQFDNVVVDGIERTARISLRLHPFDNQMLNLIPLNLIHEQPIIDVIRDYQLVAADGFVATPKVRLDKDVTIIADARSPVLARLCELSPYLHRTRILDSMTQFSPLWKINVFSSSIENAKDSIYKMAEISFTVADSVTSALSTVNVASAQQTLTVLLNSCIFRLEIDPTGSQSNFGAAISAAIMLVLFPTDEETMPTNVFDNLCNLVYNELIAWTVDRPTFVKRTGQTNAFEANVNIGGGNMNRDILAYIRFILLRRPWILYQRTYDEAYAADIFIPNIDEANINDQAYVAVNSLFSGLIQAAQRNPNPGRQISANSFRKLLKSMKDVCSNKLMPIVRLIRYNIERMARVYKWFPYSADFANRIPHFRDERLRVKVPISGVLSIMLGINKAPEAFDWYNILKFADSIRLKNYAEMESIEIIMSKAIIRNDIKPSRSKKDYIIQNLRPPTNVVAAIAKMPSATLTSILSDRILVNGVRLTQSFGVINRVIDAIRVAFENVPTAEHGIAKGALLLPYPRSFNRSSAYVRKDNVIYNAPTEVQRFNISDLLEGRFYQGLIGQVQRMSPFVINGPLQVRNLDVTAIESVTSGYLTMSSPYDACVRPEDLRHNKIVSPPTVDHYSDSNIQRPNTQFEQLLSKTSVFIIDAPKIAVQQDSTVYAFQYRDIQINTSVVDKLEFTSVKPPDVTLFNGLLVFED

SEQ ID NO: 10 - DNA, 2865 bp, Rotavirus B (equine)
  source 1..2865: mol_type = other DNA
atgatgcaccatcaccaccatcacggcagcggcctagttcctcgtggatctaggcctggagatttggatttgattacatctgcatcccaagccatacaggccactaacgataaaaatgataaacagaaaatatttgatcaactggtaaatgatctcaagaggctcagcaagggcgtcctccagccagacgttcagtctaagcttctcgaactctccaatattaacggccttgtattcaatgtggaggttcaagaagatgtcgtgaataccataaatgaccagcccgacccgaccagcatctttacacaaaacgtgtttcaacttagaacgactctctctaaaacccttttcatagatgttgacgcggaggagtattcggtatacgtgcctttggaagttcaaaacatttctccagttgtgatcgacattcgtccgattcagacatacaaacctaagactttgatgtataaagataccgctataattccgagtaacaatgatctcgttagtgatcagtggggagccgatgagatactctatgatagccacatgtttaatgacataaatatcggacaaattgaggactttgaaacgtatctgttggaaaaagccgtggaaataaaagactctctcccaaacattaaccatatctctcaattgtctaaggatactaatccgtttaatgtccataacacactgtgccttgacttcggacagaaggaatattacaaccttatctccgaccgtacgaaccagtccttcgttggccgtagacaggctgtacagttcgacaacgttgttgtcgatggtatcgagcgtactgcgagaataagcctcagacttcatccatttgataatcaaatgttgaatctgatacccctgaacctcattcacgagcagccaataattgatgttatacgtgattatcaattggtagctgcggacggttttgttgcgactcctaaagttaggctggacaaggatgtgactatcattgctgacgctcgtagcccagtgttggcgaggctgtgtgaactgagtccatatctccatcgtaccaggatacttgactcaatgacccaattctcacctttgtggaaaataaatgtcttttcctctagcattgagaatgcgaaggatagtatttacaaaatggctgaaatttctttcacggttgcagattcggtaacttccgctctttctacggttaatgtcgcatcagcgcagcaaaccctcacagtattgctgaatagttgcatttttagactcgagattgaccccacaggctcacaaagtaatttcggcgcagcgatatctgcggcaattatgttggtcctcttcccgactgatgaagaaacaatgcctacgaatgtgttcgacaacctttgcaatcttgtctataacgagcttatcgcttggacagtagatagaccaaccttcgtgaaacgcactggacagaccaacgcgtttgaggcaaatgtcaacataggtggtggaaatatgaatcgcgacatattggcctacattcgcttcatactgcttcgtaggccatggatactttaccagcgcacgtatgacgaagcttacgctgcggatatattcatccctaacatagatgaggcgaacattaatgaccaggcatatgtggcagtgaacagccttttttctggcttgatacaggctgcccaacgcaatccaaacccaggcaggcaaatttctgcgaattcctttagaaagttgcttaaatcgatgaaggatgtctgttcaaataaattgatgcctatagtccgtctgatcagatacaatatcgagaggatggcacgcgtatataaatggttcccttattctgccgacttcgcgaaccgtatcccacactttcgcgacgagagacttagagttaaagtcccgattagtggcgttctttcgataatgctcggaattaacaaagcacctgaagcgttcgactggtataatatactgaagttcgccgactcaatacgtctcaaaaattatgcagaaatggaatctatcgagatcattatgagcaaagccattatcaggaacgatatcaagccttcgcgctctaaaaaagattacattatacagaatcttaggcccccaaccaacgtagtggcagccattgcaaaaatgccgtcggccaccttgacatctatattgtcggatcgcattcttgtcaatggagtaaggttgacacaaagctttggcgtgataaacagagttattgatgccattagggttgctttcgaaaatgtgcctaccgccgagcatggaatcgcaaaaggcgcattgctcctgccttatccgagatcattcaaccgttcctctgcgtatgtaaggaaagataacgtaatttacaatgcaccgactgaagtccaaagatttaacatatccgacctccttgagggcaggttctatcagggactgataggccaagtgcagagaatgagcccgttcgtaattaacggacccttgcaggtccgcaaccttgatgttacggccatcgaaagcgttactagtggctatttgaccatgtcgagcccatatgatgcgtgtgtacgcccagaggaccttcgtcacaacaaaattgtgagcccacctacagtggatcattattctgattctaacatacaacgcccaaatacgcagtttgagcaacttctcagtaaaactagcgtcttcattattgacgcaccgaagatagcagtccagcaagattctaccgtatatgcatttcaataccgtgacattcaaataaacacgtcagtagtcgataaactcgagttcacgtccgtgaaacctccggacgtcactctgttcaacggattgcttgtatttgaagattag

SEQ ID NO: 11 - DNA, 17357 bp, synthetic construct
  source 1..17357: mol_type = other DNA
aattctactcgtaaagcgagttgaaggatcatatttagttgcgtttatgagataagattgaaagcacgtgtaaaatgtttcccgcgcgttggcacaactatttacaatgcggccaagttataaaagattctaatctgatatgttttaaaacacctttgcggcccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatgatccagcatgataagatacattgatgagtttggacaaaccacaactagaatgcagtgaaaaaaatgctttatttgtgaaatttgtgatgctattgctttatttgtaaccattataagctgcaataaacaagttccgagtttgtcagaaagcagaccaaacagcggttggaataatagcgagaacagagaaatagcggcaaaaataatacccgtatcacttttgctgatatggttgatgtcatgtagccaaatcgggaaaaacgggaagtaggctcccatgataaaaaagtaaaagaaaaagaataaaccgaacatccaaaagtttgtgttttttaaatagtacataatggatttccttacgcgaaatacgggcagacatggcctgcccggttattattatttttgacaccagaccaactggtaatggtagcgaccggcgctcagctggaattcagatctttagacataattgctcaacatatctttagtcctatcaaggaattcctgaaacagattaccaacggtttggcgattaagctttcccatgatgtcaaggatctcgcgatcatcaagaatacggttaatgatctgctcatacgttggacttccgaattcgttattagttgaaatctgtttccttacgttaagggaaaagagtgacctggttgccgtcgtcgacatttcgctaagtacttcgtgtgctatttcagggtcgatcttatatttcaaaacgtctgcgttacgctttctaagagtgctgaatgtcaaatccattggatcgaattgaatagcagccgtgtcgtcacacagcaaaccacggtcactgagatactttttgaatttaggagtcaacgtgtttgtgttttgtatatatgggagcttcatttcggtagcaagcgcaaacgtggggaagtcatgctttccctcgcgctggaggcgcgcttgcctcattgagttatatatcacacccagttcatattcttcgggcattttgttaacgatgctgacgttgggcacgtctatgttagacttgtcaataatcatcgtcgctttcacaggttcggtcttggcgactttgcgacccttcagtttggcgagcgcgtccttcgttttcgaaaaaattttcgtcacatttgaaaacagattagggagatcaccgactgaggtcacggcggagaaaaccgctgaggtaacttgttggatattcagctcggccaagattgagttgatctgatcctggagattccttatgctgacctccagtcctgaggttatgctttgtccgtatgattcgccgccggttacgaaatcaggatcagaaggcttcattacagtgtagcttatagtcatgtcgtgctccagtatcgtatcgtcgtaaccagcggcgtagcccgaacacgctccgttgcggtagtgagtgccaccaactgtgtagtcatcacctatgtagcagatataagggttaagggtgttatccacaggatagttgttgctgtccgcgtaaaattttctaccgtagcactcgaagttaaggatgatagccatacccgtgtaactaccggcgtccacttgagcgaagccgccattaatgtatctgtacttcacttttcccgccttattccttatgccagccagagagggtatataattcatgcgaaaaatgagaccaccagtattttcgagctcaccttcggccctagagtccgactggaaattttttgcgtatatagcctggttaatttgccatctgtcctcaaaataccataccgaaaatatatctcctggctgcatacagacgtactgaccattgtagtcggtagcgggacgccaggactggttgattcccgccttgcctgtcggcttgggataagtaacggttgttgcgttgactaccttgccatcccttgtataggtatacgaatactcgtatttctcaaatccgctatcaaccaaccagttcttgaacgctccgcccattgtaccatatggcttaaagtatggcttgagcttaatccaaagtgtttcagtgagaattttccaaaatccgtcttcttgttcatagattatgatcctgctggactgctctgcattagacatagttgtcctcgattgctggcaaagatcgctccaggaatacatattcagttcagtctctccacttatgtcgtagctccattccttttcgttcttaatgctgactgacgccgacagttggccgttgccatccaaaccagtgcatccattcttcgaaaaagccctagttatgagtttccactgcgcttcagtgggaaccacagacctgtcacggtaggcgtgcaggttgtagccattatcagttctaatgcaggacgtcgcttcaaagtatataccctgaactgattgtccatcgatagttatttcgccggtaacgttcaaatatattctaattactgtttgacaaggtgggtctccgttgataaaggtgtatccattagtcacttctccggaatagacctgaaactgttcagctgatacacgttcatccgcctctccaagtgcaaaacctcttacctcagttgtataattggacttgttcacttctgccttataacagtagcggccatcggccttggtaggtacatctctctcagacgtggtatcggaaccgtttcttgccgctacggcatcttcgtcttcgaaggtttcgcgcaccgtttcaccgtaactttgccactccctacgcaagtaactgattccagaacccttgtcatcgtcatccttgtaatccatcctgcagggattgtaaataaaatgtaatttacagtatagtattttaattaatatacaaatgatttgataataattcttatttaactataatatattgtgttgggttgaattaaaggtcccggcatcctcaaatgcataatttcatagtcccccttgttgtaagtgatgcgtatttctgaatctttgtaaaatagcacacaagactccaacgcgtttggcgttttattttcttgctcgactctagtttattagcggccgctcatatcttagcagcaaaggctgccaccagcattgctatttttctcgaccacacggtgtccgtatcacttgttatcatttcttgaacattccattctatttgaggtctgtttatgaatgatgtctcagggaagttggtctcaagtatatactgaacctttgaagtgcatcttgacacgaacttatagtcgttctcggtaaacgttcttctaagctcaaacatgaaggatacggcaggttgcctcggcgacgtaccattcgcccatgtcgccagggaaggcgtatccaagccattctgagatttctgttgcgtccagggggttacacgaaaccttattctggtgcacattggaaagctaaacaagcccacgccgcgttctgtatgcacgatattattgtcatcgaaaaaatcaataataaagtcgtcagccgtcatttctgcactaacatcaagaataactgactccctatttctaccctgcggcatggggaccatgatgacgccaggtgccgggaggtataccacattcaacactccgcacgtgcgagctttcaattctacgaagaacatgtgttccgcttccatgccagatacacaggttacactacctttgttgccggaccacctgccgatggcggccctgatgtttcctccgttcaagctggcaggctggcggactacgacttgctcaataatgccgacaatcagtgggttctccgtctccacaacctcaggttggatcaaagcggtgtccgtggcggcaaagttcgctatacttatctcgcttatcaacgacttcagcttcaagtcctccaccaattgttttacaacaggttcgatcacgcgtctctcaatcaccctaaccggacgtgatccctccgttctgatcgcggcggccagacagtccagtatgctctctacaccgttgcgcatatcatcgtaatcatcggttgatatacggacagccaaactgataatgggtgcatgtatggtgtatggtgacagtatggctgtttgatcgagcagtgcatacgttttgccattgacgcgcgaagctatcgcattataatcgttgaggatactctgacccgccgtattcaggtttgtgttgggagcaagactaagtacccttttctgcaatctcacacaagcattcaccgtttctatcaaatccattctagagatccgtatttataggtttttttattacaaaactgttacgaaaacagtaaaatacttatttatttgcgagatggttatcattttaattatctccatgatccaataaacctagaataaagggcccgacctttaattcaacccaacacaatatattatagttaaataagaattattatcaaatcatttgtatattaattaaaatactatactgtaaattacattttatttacaatcacagatcccgggcctaggatggcactcccattgcttctggtttttgctgcttgtgcgaaggcccaactggttataacgcccatatcaaatccagaaatatgcgtcctgcacgcctctgattggaatgtaaattccttcggcgacaattttacgaatattttcgaaacctataacagtgtaacgctctccttctatcagtacgatagcacaaactatgacgtgattgatataatctctaagagagactactcgctgtgtcacatcttggctatcgatgtaattcgtcccgaaatggatttcatcacttttctccaatccaacaacgagtgtagcaaatatgccggtcaaaagatccactaccaaaaactgtccaccaacgaagaatggttcgtatattccaagaacctgaagttttgccctctcagtgacagtctgattggtctctactgtgacacacagataaacggcacctatttccctctgtcagaaaatgaaaaatatgacgtcaccgacctcccggagtttaccgaaatgggttatgtcttttattctaacgacgatttctacatttgtaagaggatcaatgaagacaagtgggtcaattatcacctgttctatagagaatactccgcgagtggaactgtatcaagggctatatcatgggacaacgtatggacgggctttaagacatttgctcaagtagtttataagatactggacatattttttaacaatcgtagaaacttcgagcctcgtgcctaagctagcattcagaataaaacctagaggatcgatcccccggatctgatcatggagataattaaaatgataaccatctcgcaaataaataagtattttactgttttcgtaacagttttgtaataaaaaaacctataaatactaacggtaccatgatgcaccatcaccaccatcacggcagcggcctagttcctcgtggatctaggcctggagatttggatttgattacatctgcatcccaagccatacaggccactaacgataaaaatgataaacagaaaatatttgatcaactggtaaatgatctcaagaggctcagcaagggcgtcctccagccagacgttcagtctaagcttctcgaactctccaatattaacggccttgtattcaatgtggaggttcaagaagatgtcgtgaataccataaatgaccagcccgacccgaccagcatctttacacaaaacgtgtttcaacttagaacgactctctctaaaacccttttcatagatgttgacgcggaggagtattcggtatacgtgcctttggaagttcaaaacatttctccagttgtgatcgacattcgtccgattcagacatacaaacctaagactttgatgtataaagataccgctataattccgagtaacaatgatctcgttagtgatcagtggggagccgatgagatactctatgatagccacatgtttaatgacataaatatcggacaaattgaggactttgaaacgtatctgttggaaaaagccgtggaaataaaagactctctcccaaacattaaccatatctctcaattgtctaaggatactaatccgtttaatgtccataacacactgtgccttgacttcggacagaaggaatattacaaccttatctccgaccgtacgaaccagtccttcgttggccgtagacaggctgtacagttcgacaacgttgttgtcgatggtatcgagcgtactgcgagaataagcctcagacttcatccatttgataatcaaatgttgaatctgatacccctgaacctcattcacgagcagccaataattgatgttatacgtgattatcaattggtagctgcggacggttttgttgcgactcctaaagttaggctggacaaggatgtgactatcattgctgacgctcgtagcccagtgttggcgaggctgtgtgaactgagtccatatctccatcgtaccaggatacttgactcaatgacccaattctcacctttgtggaaaataaatgtcttttcctctagcattgagaatgcgaaggatagtatttacaaaatggctgaaatttctttcacggttgcagattcggtaacttccgctctttctacggttaatgtcgcatcagcgcagcaaaccctcacagtattgctgaatagttgcatttttagactcgagattgaccccacaggctcacaaagtaatttcggcgcagcgatatctgcggcaattatgttggtcctcttcccgactgatgaagaaacaatgcctacgaatgtgttcgacaacctttgcaatcttgtctataacgagcttatcgcttggacagtagatagaccaaccttcgtgaaacgcactggacagaccaacgcgtttgaggcaaatgtcaacataggtggtggaaatatgaatcgcgacatattggcctacattcgcttcatactgcttcgtaggccatggatactttaccagcgcacgtatgacgaagcttacgctgcggatatattcatccctaacatagatgaggcgaacattaatgaccaggcatatgtggcagtgaacagccttttttctggcttgatacaggctgcccaacgcaatccaaacccaggcaggcaaatttctgcgaattcctttagaaagttgcttaaatcgatgaaggatgtctgttcaaataaattgatgcctatagtccgtctgatcagatacaatatcgagaggatggcacgcgtatataaatggttcccttattctgccgacttcgcgaaccgtatcccacactttcgcgacgagagacttagagttaaagtcccgattagtggcgttctttcgataatgctcggaattaacaaagcacctgaagcgttcgactggtataatatactgaagttcgccgactcaatacgtctcaaaaattatgcagaaatggaatctatcgagatcattatgagcaaagccattatcaggaacgatatcaagccttcgcgctctaaaaaagattacattatacagaatcttaggcccccaaccaacgtagtggcagccattgcaaaaatgccgtcggccaccttgacatctatattgtcggatcgcattcttgtcaatggagtaaggttgacacaaagctttggcgtgataaacagagttattgatgccattagggttgctttcgaaaatgtgcctaccgccgagcatggaatcgcaaaaggcgcattgctcctgccttatccgagatcattcaaccgttcctctgcgtatgtaaggaaagataacgtaatttacaatgcaccgactgaagtccaaagatttaacatatccgacctccttgagggcaggttctatcagggactgataggccaagtgcagagaatgagcccgttcgtaattaacggacccttgcaggtccgcaaccttgatgttacggccatcgaaagcgttactagtggctatttgaccatgtcgagcccatatgatgcgtgtgtacgcccagaggaccttcgtcacaacaaaattgtgagcccacctacagtggatcattattctgattctaacatacaacgcccaaatacgcagtttgagcaacttctcagtaaaactagcgtcttcattattgacgcaccgaagatagcagtccagcaagattctaccgtatatgcatttcaataccgtgacattcaaataaacacgtcagtagtcgataaactcgagttcacgtccgtgaaacctccggacgtcactctgttcaacggattgcttgtatttgaagattagggatccggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgcggcgtgttaacatacaagttgctaaccggcggccgacacccatttgaaaaaagcgaagacgaaatgttggacttgaatagcatgaagcgtcgtcagcaatacaatgacattggcgttttaaaacacgttcgtaacgttaacgctcgtgactttgtgtactgcctaacaagatacaacatagattgtagactcacaaattacaaacaaattataaaacatgagtttttgtcgtaaaaatgccacttgttttacgagtagaattcccagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcggacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

SEQ ID NO: 12 - DNA, 11929 bp, synthetic construct
  source 1..11929: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttcttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacttgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatcccggtccgaagcgcgcggaattcatggatttgatagaaacggtgaatgcttgtgtgagattgcagaaaagggtacttagtcttgctcccaacacaaacctgaatacggcgggtcagagtatcctcaacgattataatgcgatagcttcgcgcgtcaatggcaaaacgtatgcactgctcgatcaaacagccatactgtcaccatacaccatacatgcacccattatcagtttggctgtccgtatatcaaccgatgattacgatgatatgcgcaacggtgtagagagcatactggactgtctggccgccgcgatcagaacggagggatcacgtccggttagggtgattgagagacgcgtgatcgaacctgttgtaaaacaattggtggaggacttgaagctgaagtcgttgataagcgagataagtatagcgaactttgccgccacggacaccgctttgatccaacctgaggttgtggagacggagaacccactgattgtcggcattattgagcaagtcgtagtccgccagcctgccagcttgaacggaggaaacatcagggccgccatcggcaggtggtccggcaacaaaggtagtgtaacctgtgtatctggcatggaagcggaacacatgttcttcgtagaattgaaagctcgcacgtgcggagtgttgaatgtggtatacctcccggcacctggcgtcatcatggtccccatgccgcagggtagaaatagggagtcagttattcttgatgttagtgcagaaatgacggctgacgactttattattgattttttcgatgacaataatatcgtgcatacagaacgcggcgtgggcttgtttagctttccaatgtgcaccagaataaggtttcgtgtaaccccctggacgcaacagaaatctcagaatggcttggatacgccttccctggcgacatgggcgaatggtacgtcgccgaggcaacctgccgtatccttcatgtttgagcttagaagaacgtttaccgagaacgactataagttcgtgtcaagatgcacttcaaaggttcagtatatacttgagaccaacttccctgagacatcattcataaacagacctcaaatagaatggaatgttcaagaaatgataacaagtgatacggacaccgtgtggtcgagaaaaatagcaatgctggtggcagcctttgctgctaagatatgataaaacttgtttattgcagcttataatggttacaaataaagcaatagcatcacaaatttcacaaataaagcatttttttcactgcattctagttgtggtttgtccaaactcatcaatgtatcttatcatgtctggatccgtatactccggaatattaatagatcatggagataattaaaatgataaccatctcgcaaataaataagtattttactgttttcgtaacagttttgtaataaaaaaacctataaatatggattacaaggatgacgatgacaagggttctggaaggacaactatgtctaatgcagagcagtccagcaggatcataatctatgaacaagaagacggattttggaaaattctcactgaaacactttggattaagctcaagccatactttaagccatatggtacaatgggcggagcgttcaagaactggttggttgatagcggatttgagaaatacgagtattcgtatacctatacaagggatggcaaggtagtcaacgcaacaaccgttacttatcccaagccgacaggcaaggcgggaatcaaccagtcctggcgtcccgctaccgactacaatggtcagtacgtctgtatgcagccaggagatatattttcggtatggtattttgaggacagatggcaaattaaccaggctatatacgcaaaaaatttccagtcggactctagggccgaaggtgagctcgaaaatactggtggtctcatttttcgcatgaattatataccctctctggctggcataaggaataaggcgggaaaagtgaagtacagatacattaatggcggcttcgctcaagtggacgccggtagttacacgggtatggctatcatccttaacttcgagtgctacggtagaaaattttacgcggacagcaacaactatcctgtggataacacccttaacccttatatctgctacataggtgatgactacacagttggtggcactcactaccgcaacggagcgtgttcgggctacgccgctggttacgacgatacgatactggagcacgacatgactataagctacactgtaatgaagccttctgatcctgatttcgtaaccggcggcgaatcatacggacaaagcataacctcaggactggaggtcagcataaggaatctccaggatcagatcaactcaatcttggccgagctgaatatccaacaagttacctcagcggttttctccgccgtgacctcagtcggtgatctccctaatctgttttcaaatgtgacgaaaattttttcgaaaacgaaggacgcgctcgccaaactgaagggtcgcaaagtcgccaagaccgaacctgtgaaagcgacgatgattattgacaagtctaacatagacgtgcccaacgtcagcatcgttaacaaaatgcccgaagaatatgaactgggtgtgatatataactcaatgaggcaagcgcgcctccagcgcgagggaaagcatgacttccccacgtttgcgcttgctaccgaaatgaagctcccatatatacaaaacacaaacacgttgactcctaaattcaaaaagtatctcagtgaccgtggtttgctgtgtgacgacacggctgctattcaattcgatccaatggatttgacattcagcactcttagaaagcgtaacgcagacgttttgaaatataagatcgaccctgaaatagcacacgaagtacttagcgaaatgtcgacgacggcaaccaggtcactcttttcccttaacgtaaggaaacagatttcaactaataacgaattcggaagtccaacgtatgagcagatcattaaccgtattcttgatgatcgcgagatccttgacatcatgggaaagcttaatcgccaaaccgttggtaatctgtttcaggaattccttgataggactaaagatatgttgagcaattatgtctaatgaaacttgtttattgcagcttataatggttacaaataaagcaatagcatcacaaatttcacaaataaagcatttttttcactgcattctagttgtggtttgtccaaactcatcaatgtatcttatcatgtctggatccgtatactccggaatattaatagatcatggagataattaaaatgataaccatctcgcaaataaataagtattttactgttttcgtaacagttttgtaataaaaaaacctataaatatggcactcccattgcttctggtttttgctgcttgtgcgaaggcccaactggttataacgcccatatcaaatccagaaatatgcgtcctgcacgcctctgattggaatgtaaattccttcggcgacaattttacgaatattttcgaaacctataacagtgtaacgctctccttctatcagtacgatagcacaaactatgacgtgattgatataatctctaagagagactactcgctgtgtcacatcttggctatcgatgtaattcgtcccgaaatggatttcatcacttttctccaatccaacaacgagtgtagcaaatatgccggtcaaaagatccactaccaaaaactgtccaccaacgaagaatggttcgtatattccaagaacctgaagttttgccctctcagtgacagtctgattggtctctactgtgacacacagataaacggcacctatttccctctgtcagaaaatgaaaaatatgacgtcaccgacctcccggagtttaccgaaatgggttatgtcttttattctaacgacgatttctacatttgtaagaggatcaatgaagacaagtgggtcaattatcacctgttctatagagaatactccgcgagtggaactgtatcaagggctatatcatgggacaacgtatggacgggctttaagacatttgctcaagtagtttataagatactggacatattttttaacaatcgtagaaacttcgagcctcgtgcctaatgaaacttgtttattgcagcttataatggttacaaataaagcaatagcatcacaaatttcacaaataaagcatttttttcactgcattctagttgtggtttgtccaaactcatcaatgtatcttatcatgtctggatccgtatactccggaatattaatagatcatggagataattaaaatgataaccatctcgcaaataaataagtattttactgttttcgtaacagttttgtaataaaaaaacctataaatatgatgcaccatcaccaccatcacggcagcggcctagttcctcgtggatctaggcctggagatttggatttgattacatctgcatcccaagccatacaggccactaacgataaaaatgataaacagaaaatatttgatcaactggtaaatgatctcaagaggctcagcaagggcgtcctccagccagacgttcagtctaagcttctcgaactctccaatattaacggccttgtattcaatgtggaggttcaagaagatgtcgtgaataccataaatgaccagcccgacccgaccagcatctttacacaaaacgtgtttcaacttagaacgactctctctaaaacccttttcatagatgttgacgcggaggagtattcggtatacgtgcctttggaagttcaaaacatttctccagttgtgatcgacattcgtccgattcagacatacaaacctaagactttgatgtataaagataccgctataattccgagtaacaatgatctcgttagtgatcagtggggagccgatgagatactctatgatagccacatgtttaatgacataaatatcggacaaattgaggactttgaaacgtatctgttggaaaaagccgtggaaataaaagactctctcccaaacattaaccatatctctcaattgtctaaggatactaatccgtttaatgtccataacacactgtgccttgacttcggacagaaggaatattacaaccttatctccgaccgtacgaaccagtccttcgttggccgtagacaggctgtacagttcgacaacgttgttgtcgatggtatcgagcgtactgcgagaataagcctcagacttcatccatttgataatcaaatgttgaatctgatacccctgaacctcattcacgagcagccaataattgatgttatacgtgattatcaattggtagctgcggacggttttgttgcgactcctaaagttaggctggacaaggatgtgactatcattgctgacgctcgtagcccagtgttggcgaggctgtgtgaactgagtccatatctccatcgtaccaggatacttgactcaatgacccaattctcacctttgtggaaaataaatgtcttttcctctagcattgagaatgcgaaggatagtatttacaaaatggctgaaatttctttcacggttgcagattcggtaacttccgctctttctacggttaatgtcgcatcagcgcagcaaaccctcacagtattgctgaatagttgcatttttagactcgagattgaccccacaggctcacaaagtaatttcggcgcagcgatatctgcggcaattatgttggtcctcttcccgactgatgaagaaacaatgcctacgaatgtgttcgacaacctttgcaatcttgtctataacgagcttatcgcttggacagtagatagaccaaccttcgtgaaacgcactggacagaccaacgcgtttgaggcaaatgtcaacataggtggtggaaatatgaatcgcgacatattggcctacattcgcttcatactgcttcgtaggccatggatactttaccagcgcacgtatgacgaagcttacgctgcggatatattcatccctaacatagatgaggcgaacattaatgaccaggcatatgtggcagtgaacagccttttttctggcttgatacaggctgcccaacgcaatccaaacccaggcaggcaaatttctgcgaattcctttagaaagttgcttaaatcgatgaaggatgtctgttcaaataaattgatgcctatagtccgtctgatcagatacaatatcgagaggatggcacgcgtatataaatggttcccttattctgccgacttcgcgaaccgtatcccacactttcgcgacgagagacttagagttaaagtcccgattagtggcgttctttcgataatgctcggaattaacaaagcacctgaagcgttcgactggtataatatactgaagttcgccgactcaatacgtctcaaaaattatgcagaaatggaatctatcgagatcattatgagcaaagccattatcaggaacgatatcaagccttcgcgctctaaaaaagattacattatacagaatcttaggcccccaaccaacgtagtggcagccattgcaaaaatgccgtcggccaccttgacatctatattgtcggatcgcattcttgtcaatggagtaaggttgacacaaagctttggcgtgataaacagagttattgatgccattagggttgctttcgaaaatgtgcctaccgccgagcatggaatcgcaaaaggcgcattgctcctgccttatccgagatcattcaaccgttcctctgcgtatgtaaggaaagataacgtaatttacaatgcaccgactgaagtccaaagatttaacatatccgacctccttgagggcaggttctatcagggactgataggccaagtgcagagaatgagcccgttcgtaattaacggacccttgcaggtccgcaaccttgatgttacggccatcgaaagcgttactagtggctatttgaccatgtcgagcccatatgatgcgtgtgtacgcccagaggaccttcgtcacaacaaaattgtgagcccacctacagtggatcattattctgattctaacatacaacgcccaaatacgcagtttgagcaacttctcagtaaaactagcgtcttcattattgacgcaccgaagatagcagtccagcaagattctaccgtatatgcatttcaataccgtgacattcaaataaacacgtcagtagtcgataaactcgagttcacgtccgtgaaacctccggacgtcactctgttcaacggattgcttgtatttgaagattagtaaaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg